(19) United States
(12) Patent Application Publication
(10) Pub. No.: US 2012/0266892 A1
(43) Pub. Date: Oct. 25, 2012

(54) TETHERED IMPLANT AND RELATED METHOD OF USE

(75) Inventors: Matthew LaPlaca, Cumberland, RI (US); David Callaghan, Mansfield, MA (US); Jeffrey Model, Cambridge, MA (US); James Duronio, Westford, MA (US); Mark Putnam, Weymouth, MA (US)

(73) Assignee: Hologic, Inc., Marlborough, MA (US)

(21) Appl. No.: 13/091,881

(22) Filed: Apr. 21, 2011

Related U.S. Application Data
(63) Continuation-in-part of application No. 13/026,925, now abandoned.

(51) Int. Cl. A61F 6/06

(52) U.S. Cl. 128/831

(57) ABSTRACT

An implant configured for insertion and placement within a fallopian tube of a female during a sterilization procedure. The implant includes an implant body having proximal and distal ends and a tether. The tether is attached to the implant body and extends a distance away from the implant body. The tether may be bioabsorbable and/or detachably coupled to the implant body so that the tether can be separated from the implant body without dislodging the implant body from the fallopian tube.

### FIELD OF THE INVENTION

The present disclosure generally relates to the field of hysteroscopic sterilization and, more particularly, to implants adapted for insertion and placement within the fallopian tubes during a hysteroscopic sterilization procedure.

### BACKGROUND OF THE INVENTION

It is sometimes desirable to close the fallopian tubes of a female for sterilization purposes or for other medical related reasons. A common sterilization method is tubal ligation, a surgical procedure in which the fallopian tubes are accessed via an incision, then cut and tied or clamped to prevent future pregnancies.

Another sterilization method involves inserting an implant into each fallopian tube. The implants are delivered to the fallopian tubes hysteroscopically with the aid of a handheld delivery catheter, thus avoiding the need to undergo a more invasive tubal ligation procedure. With the implants in place, the fallopian tubes become occluded over a period of time.

To confirm the fallopian tubes are occluded, a physician will usually perform a hysterosalpingogram (HSG) about three months after the implants are placed. An HSG involves pressurizing the uterus with radiopaque fluid while taking a real-time fluoroscopic image. The HSG test is a radiology procedure usually done in the radiology department of a hospital in which radiopaque fluid (dye) is injected into the uterine cavity through the vagina and cervix. The uterine cavity fills with dye and if the fallopian tubes are open (i.e. not occluded), the dye will then fill the tubes and spill out into the abdominal cavity. In this way, it may be determined whether the fallopian tubes are open or occluded and where the occlusion is located.

It would be desirable to provide implants and related methods in which implant placement and/or effectiveness may be determined by direct visualization and without the need for HSG procedures.

### SUMMARY

In one aspect, an implant for use in a female sterilization procedure is disclosed. The implant comprises an implant body configured for insertion and placement within a fallopian and a tether. The implant body defines a proximal end and a distal end. The tether is attached to the implant body and extends away from the implant body. In certain embodiments, the tether provides a visible marker leading to the implant body to confirm the presence and location of the implant body.

In certain embodiments, the tether is detachably coupled to the implant body and is adapted to detach from the implant body when a predetermined threshold force is applied to the tether. And in some such embodiments, the implant further includes a frangible interface between the implant body and the tether, and the tether is adapted to detach from the implant body at the frangible interface when a predetermined threshold force is applied to the tether.

In another aspect, a method for determining the location and/or placement of an implanted implant is described, wherein the implant is intended to be implanted in a desired location within a fallopian tube hysteroscopically using an implant delivery device. The method comprises the steps of: providing an implanted implant, the implant including an implant body defining a proximal end and a distal end, and a tether attached to the implant body and extending a distance from the implant body; visually locating at least a portion of the tether with a hysteroscope; and determining the location and/or placement of the implant body relative to a desired location within a fallopian tube. In one embodiment, the desired location is the uterotubal junction.

In one embodiment, the method further comprises the steps of applying a predetermined threshold force to the tether and substantially simultaneously detaching the tether from the implant body.

These and other features, aspects, and advantages of the disclosed embodiments will become better understood with regard to the following description, appended claims, and accompanying drawings.

### BRIEF DESCRIPTION OF DRAWINGS

FIG. 1 is a partial view of a female reproductive system.

FIG. 2 is a partial cross-sectional view of a fallopian tube of the female reproductive system.

FIG. 3 is an enlarged side view of an implant described in the present disclosure.

FIG. 4 is a cross-sectional view of a fallopian tube of the reproductive system with the implant of FIG. 3 implanted therein.

FIG. 5 is an enlarged front-side perspective view of an implant described in the present disclosure having a substantially solid core.

FIG. 6A is an enlarged side view of an implant described in the present disclosure with the tether frangibly attached to the implant body.

FIG. 6B is an enlarged side view of the implant described in FIG. 6A with the tether detached from the implant body upon application of a predetermined threshold force F.

FIG. 7A is an enlarged side view of an implant described in the present disclosure with the tether defining a frangible point along its length between a first segment and a second segment.

FIG. 7B is an enlarged side view of the implant described in FIG. 7A with the second segment of the tether detached from the first segment upon application of a predetermined threshold force F.

FIG. 8 is a flow chart describing steps associated with a method for confirming the location and placement of the implants of FIGS. 3, 5, 6A-B and 7A-B upon implantation within a fallopian tube as shown, for example, in FIG. 4.

FIG. 9 is a partial view of a female reproductive system with a hysteroscope inserted through the vagina and into the uterus, the hysteroscope being utilized in the method outlined in FIG. 7 to locate and confirm placement of implants within fallopian tubes.

Although the drawings represent embodiments of the present invention, the drawings are not necessarily to scale and certain features may be exaggerated in order to better illustrate and explain the present invention. The exemplification set out herein illustrates certain embodiments of the invention, in one, or more forms, and such exemplifications are not to be construed as limiting the scope of the invention in any manner.

### DETAILED DESCRIPTION

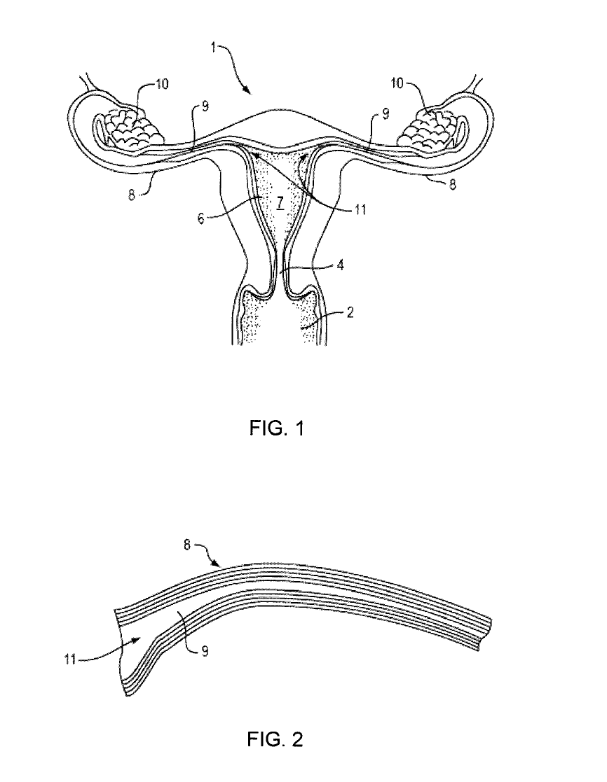

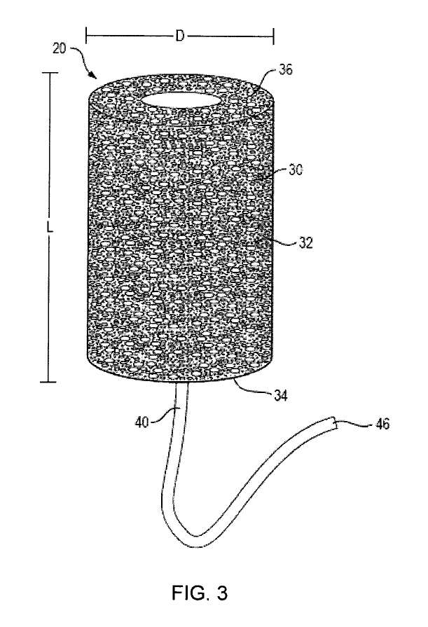

FIGS. 1-2 generally illustrate some of the major elements of the female reproductive system 1. The reproductive system 1 includes the vagina 2, cervix 4, uterus 6, fallopian tubes 8 and ovaries 10. The uterus 6 defines a cavity referred to as the uterine cavity 7. The cervix 4 forms the lower neck of the uterus 6 and is the junction between the vagina 2 and the uterus 6. The fallopian tubes 8 are hollow organs, each defining an internal lumen 9 that extends therethrough which connects the uterus 6 to the ovaries 10. The proximal end of each fallopian tube 8 (i.e. where the tube joins the uterine cavity 7) is often referred to as the uterotubal junction 11. The uterotubal junction 11 is a generally tubular-shaped section about 10 mm in length and may serve as a location where the implants described herein are disposed during a sterilization procedure

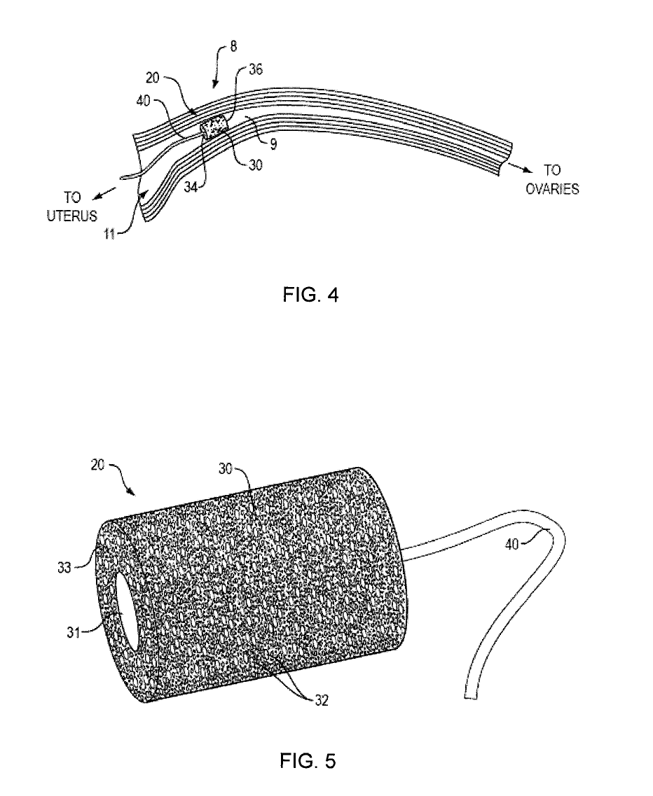

Referring now to FIG. 3, an implant embodying the present invention is indicated generally by the reference numeral 20. The implant 20 is configured for insertion and placement via a delivery device (not shown) within a fallopian tube as part of a hysteroscopic sterilization procedure. Related sterilizations procedures and implant delivery devices are described in: U.S. patent application Ser. No. 12/692,057, entitled “Sterilization Device and Method”; U.S. patent application Ser. No. 12/773,332, entitled “Radiopaque Implant”, U.S. Patent Application Publication No. 2009-0036840, entitled “Atraumatic Ball Tip and Side Wall Opening”; U.S., Patent Application Publication Nos. 2007-0215163 and 2004-0255958, both entitled “Method and Apparatus for Tubal Occlusion”; U.S. patent application Ser. No. 11/562,882, entitled “Delivery Catheter with Implant Ejection Mechanism”; U.S. Pat. Nos. 7,582,085 and 6,780,182, both entitled “Catheter Placement Detection System and Operator Interface”; U.S. Pat. Nos. 7,220,259, 6,726,682, 6,712,810, 6,346,102, 6,309,384, 6,068,626, and 5,954,715, each entitled “Method and Apparatus for Tubal Occlusion; U.S. Pat. No. 5,681,572, entitled “Porous Material Produce and Process”; and U.S. Pat. No. 5,095,917, entitled “Transuterine Sterilization Apparatus and Method”; all of which are incorporated herein by references in their entireties as part of the present disclosure.

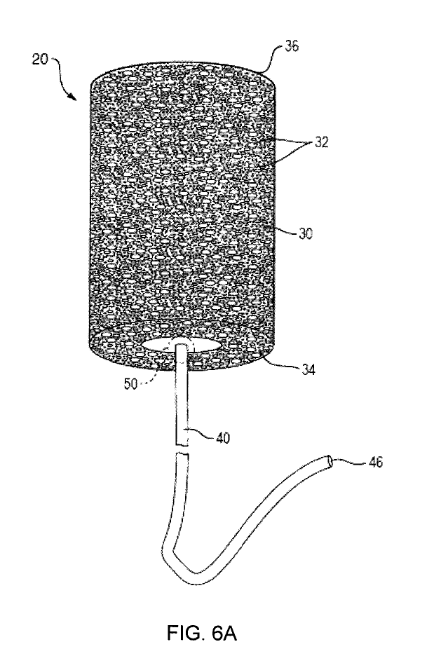

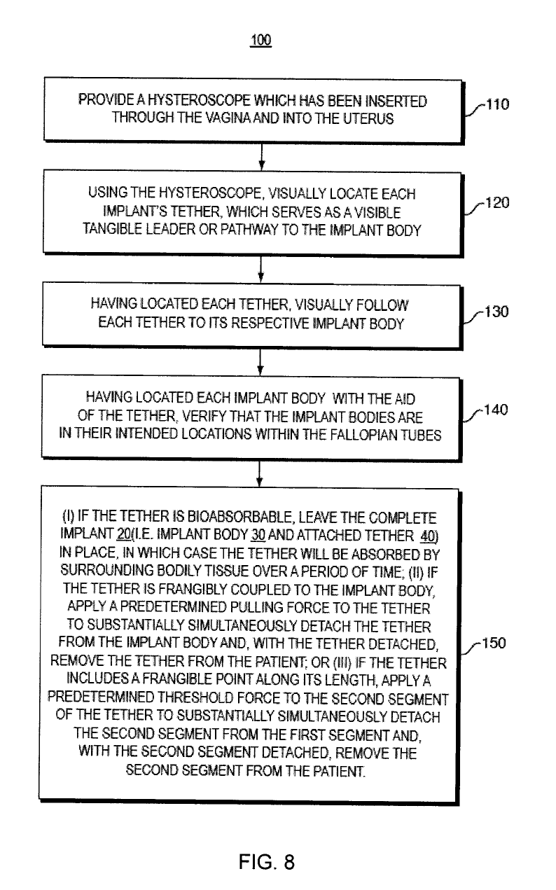

The implant 20 comprises an implant body 30 and a tether 40 (described in further detail below) extending therefrom. The implant body 30 defines a plurality of pores 32 and is typically formed as a matrix or plug having a pore size chemistry and architecture which may facilitate cellular ingrowth into the material over time. The implant body 30 further defines a longitudinal axis, as well as a proximal end 34 and a distal end 36 respectively located at opposite ends of the longitudinal axis. When inserted into a fallopian tube 8 (see e.g. FIGS. 4 and 8), the implant body 30 is oriented so that its proximal end 34 faces toward the uterus 6 while the distal end 36 faces toward the ovary 10 (i.e. away from the uterus). In the illustrated embodiment, the implant body 30 is an elongate body having an approximately cylindrical configuration. However, as one skilled in the art would appreciate, the implant body 30 can take on any suitable configuration that facilitates and enables fallopian tube occlusion. In terms of dimensions, the implant body 30 may have, in one embodiment, an outer diameter D ranging between about 1.0 mm and about 3.0 mm and, more preferably, may have an outer diameter ranging between about 1.4 mm and about 1.8 mm. Additionally, the implant body 30 may have, in one embodiment, an overall length L ranging between about 2 mm and about 10 mm and, more preferably, may have a an overall length ranging between about 3 mm and about 6 mm.

In certain embodiments, the implant body 30 is made of ePTFE (also referred to as expanded Teflon or expanded polytetraflouroethylene), porous silicone, acrylic copolymer, cellulose acetate, polyethylene and high density polyethylene (HDPE), PE, polyester, and sintered, micro-knurled, or molded titanium and platinum. Textured polyamides or polyimides, hydroxyapitite, and hydrogels are also potential suitable materials. Preferably, these materials are formed into a foamed material, which is molded or otherwise formed into the implant body 30. The preferable pore sizes of the foam fall into the two distinct ranges, namely a 1-20 micron pore size or a 40-200 micron pore size. The foam is preferably formed as a reticulated foam, meaning that the pores 32 communicate with other pores, rather than existing as discrete and isolated voids within the material. In one embodiment, the implant body 30 is made from silicone foam having a pore size ranging between about 50 and about 150 microns. Silicone foam is readily formed into porous implants with the procedure set forth, for example, in U.S. Pat. No. 5,605,693, entitled “Method of Marking a Porous Implant”, which is incorporated by reference in its entirety as part of the present disclosure.

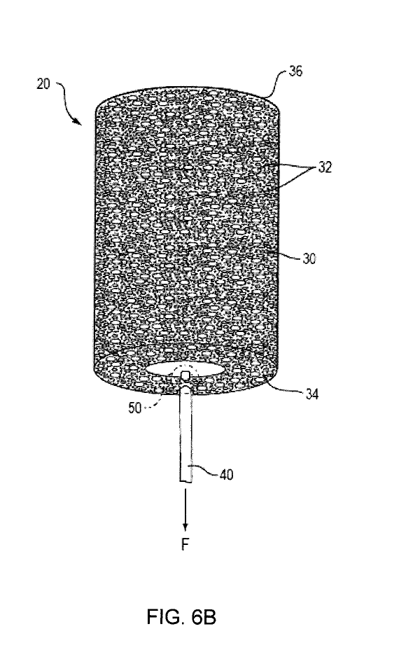

In the embodiment of FIG. 5, the implant body 30 has a substantially solid core 31 (i.e. the core 31 may define one or more pores therein) surrounded by a foam or a porous material 33 having a reticulated network of pores. The tether 40 (described below) may be attached (frangibly or otherwise) to the core 31, porous mater 33 or both. As recognized by those skilled in the art, the implant body 30 may take on numerous cross-sectional configurations including, but not limited to, multiple layers including multiple solid and foam layers, foam layers having different pore sizes, multiple solid layers having differing molecular configurations, foam and/or solid layers comprising different materials, or multiple layers of varying thicknesses. As an alternative to having layers, the implant body 30 may be formed as a single porous foam structure (i.e. no core or layers).

The implant 20 further includes a tether 40 attached or otherwise affixed to the implant body 30. In at least one aspect the tether serves as a marker or tangible guide leading from the tether to the implant body to confirm the presence and location of the implant body 30. In the illustrated embodiments, the tether 40 is affixed to the proximal end 34 of the implant body 30; however, it should be noted that the tether 40 may be affixed to any portion of the implant body 30 both internally and externally relative to an outer surface of the implant body. The tether 40 is attached or otherwise affixed to the implant body 30 using any of numerous methods known to those skilled in the art. For example, the tether 40 could be glued, bonded, ultrasonically welded, mechanically coupled or overmolded to the implant body. In certain embodiments, the tether 40 is typically monofilament or multifilament (braided) suture or thread-like member having a length (i.e. the distance measured from the point on the surface of the implant body 30 from which the tether 40 extends to a terminal end 46) of at least about 5 mm and, in some embodiments, a length between about 5 mm and about 10 mm. The tether 40 is made from any suitable material such as, for example, from a bioabsorbable material such as polyglycolide (polyglycolic acid), polyglactic acid, polyglyconate or polydioxanone, or a non-bioabsorbable material such as nylon, silk, polypropylene, polyethylene, polyethylene terephthalate (PET), polybutester or braided polyester. In one embodiment, the tether additionally comprises a radiopaque material to provide visualization means under fluoroscopic techniques known in the art. Such radiopaque material may be, for example, within the tether material itself, or may be in the form of bands or other markings at one or more locations along the length of the tether. It should be noted that the above list of materials is not exhaustive and, therefore, other suitable materials known to those skilled in the art including variations of the above-listed materials may be used alone or in combination with any of the above listed materials.

In one embodiment, the tether 40 is fabricated from a bioabsorbable material and attached to the implant body 30 as described above. Having this configuration, the tether 40 is adapted to be absorbed by surrounding tissue after a period of time following implantation into a respective fallopian tube. The period of time from implantation to full absorption is, to a large extent, predicated on the material used to fabricate the tether; however, in certain embodiments full absorption of the tether 40 will occur in a time period ranging from about 1 month to about 6 months.

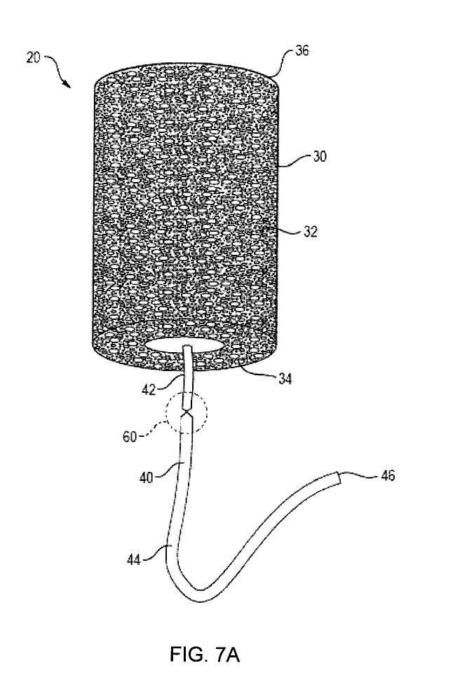

In another embodiment, shown in FIGS. 6A-B, the tether 40 is formed from a non-bioabsorbable material and is frangibly or otherwise detachably coupled to the implant body 30 so that the tether can be detached and removed from the implant body upon application of a predetermined threshold force F. In this manner the tether 40 will detach from the implant body 30 at a frangible or break-away interface 50. The interface 50 is located between a portion of the tether 40 and the implant body 30 at the point or region where the tether and implant body are attached. Depending on the manner in which the tether 40 is attached to the implant body 30, the frangible or break-away interface 50 may be located internally relative to the implant body mass or at a location externally relative to the implant body mass. The frangible or break-away interface 50 may be defined, for example, by an adhesive bond or mechanical connection between the tether 40 and implant body 30 that is designed to fail upon application of a threshold force. However, it should be noted that other suitable configurations enabling the tether 50 to detach form the implant body 30 upon application of a predetermined threshold may be used. Using embodiments in which the tether 40 is made from a bioabsorbable material gives the physician the option to either manually detach and remove the tether 40 from the patient or leave the tether 40 attached to the implant body 30, in which case the tether 40 will be absorbed by the surrounding bodily tissue over a period of time.

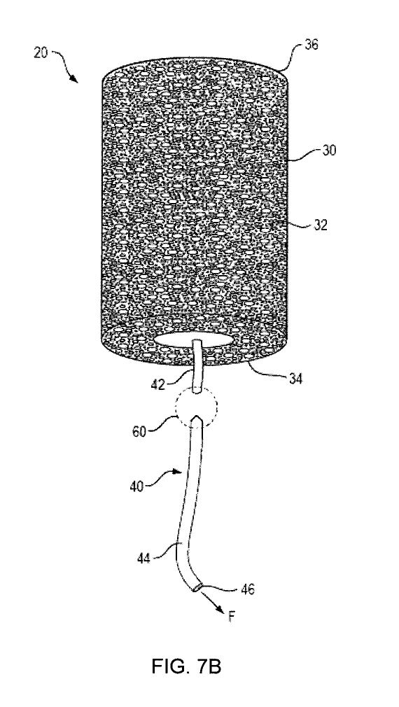

In yet another embodiment, shown in FIGS. 7A-B, the tether 40 is formed from a non-bioabsorbable material and defines a frangible break-away point or segment 60 at a predetermine location along its length, thereby allowing a portion of the tether to break off when a predetermined threshold force is applied. In the illustrated embodiment, the frangible segment 60 is a reduced diameter neck; however, it should be noted that other suitable frangible configurations known to those skilled in the art may be employed. The tether 40 further defines a first segment 42, a second segment 44, and a terminal end 46. In a preferred embodiment, the second segment 44 is longer than the first segment 42. The first segment 42 extends from the implant body 30 to the frangible break-away segment 60, and the second segment 44 extends from the frangible break-away segment 60 to the terminal end 46. Having this configuration, the second segment 44 is detachable from the first segment 42 at the frangible break-away segment 60 upon application of a predetermined threshold force F (i.e. the tether 40 will break at the frangible break-away point upon application of a predetermined threshold force). The first segment 42 will remain attached to the implant body 30. In this embodiment, it is further conceivable that he first segment 42 could be fabricated from a bioabsorbable material and, thus, will be absorbed by the surrounding bodily tissue over a period of time.

In the above-described embodiments, the predetermined threshold force F is of a magnitude selected to substantially avoid dislodging a correctly placed implant body 30 from a fallopian tube when the force is applied. In some embodiments, the threshold force F is a tensile or pulling force in the range of about 0.5 lbf (pound-force) to about 1.5 lbf and, more preferably, in the range of about 0.8 lbf to about 1.0 Ibf. However, as appreciated by those skilled in the art, the magnitude of the threshold force could be adjusted above or below the aforementioned ranges to an amount best suited to the materials used and to the manner by which the tether is detached. Additionally, the threshold force could be adjusted as necessary based upon the methods and devices used to deliver the implants to avoid moving the implants within the fallopian tubes and interfering with the tubal occlusion process.

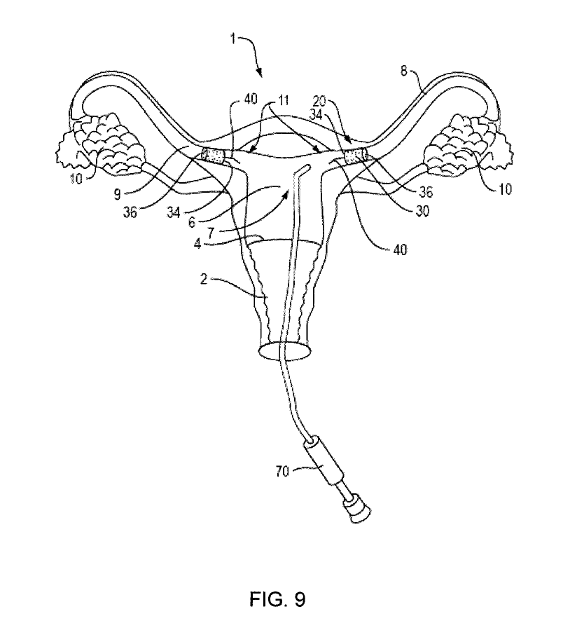

Drawing attention to FIGS. 8-9, a method 100 for confirming placement of an implant 20 within a fallopian tube is described. At step 110, a hysteroscope 70 including a lumination source (not shown) is provided, which has been inserted through the vagina 2 and into the uterine cavity 7 of a female patient. At step 120, the physician, with the aid of the hysteroscope, visually locates the implant's tether 40. The tether 40 serves as a visible tangible leader or pathway to the implant body 30. At step 130, the physician, having located the tether 40, verifies that the implant body 30 is in the desired location within a fallopian tube 8. Such verification is made by visualizing the tether itself (with or without the use of a marking system on the tether 40, such as colored markings or graduation markings at known locations on the tether), or alternatively by visually following the tether to its respective implant body 30 as shown at step 140. In one embodiment, the desired location is the uterotubal junction 11.

Moving on to step 150, with the implant body 30 confirmed in its desired location, one of the following scenarios may occur: (i) if the tether 40 is bioabsorbable, the physician may leave the complete implant 20 (i.e. implant body 30 and attached tether 40) in place, in which case the tether will be absorbed by surrounding bodily tissue over a period of time; (ii) if the tether 40 is frangibly coupled to the implant body 30, the physician may apply a predetermined pulling force to the tether to substantially simultaneously detach the tether 40 from the implant body and remove the tether from the patient; or (iii) if the tether 40 includes a frangible point 60 along its length, the physician may apply a predetermined threshold force to the second segment 44 of the tether 40 to substantially simultaneously detach the second segment 44 from the first segment 42 of the tether 40 and remove the second segment 44 from the patient.

The above-describe tethered implants and related methods advantageously provide a feasible and cost effective way to verify that that one or more implants delivered to the fallopian tubes during a hysteroscopic sterilization procedure were placed in the intended location. The verification can occur immediately following implantation utilizing the same hysteroscope used to guide the implant delivery device to the fallopian tubes. In this manner, the patient does not have to undergo a subsequent imaging procedure to verify proper placement of the implants, nor does the patient have to wait 3-6 months to verify placement at the time the hysterosalpingogram (HSG) is performed. Additionally, the tethers may advantageously serve as markers, which during the course of a sterilization procedure, provide visual confirmation to the physician as to which fallopian tube received the first implant; and after the procedure is complete, provide visual confirmation that both fallopian tubes received implants.

As may be recognized by those of ordinary skill in the pertinent art based on the teachings herein, numerous changes and modifications may be made to the above-described and other embodiments of the present invention without departing from the spirit and scope of the invention as defined and/or described in the specification, drawings and appended claims. It should be understood that the embodiments shown and described and all changes, modifications and equivalents that come within the spirit and scope of the invention are desired to be protected. Accordingly, this disclosure is to be taken in an illustrative, as opposed to a limiting sense.

All terms used in the claims are intended to be given their broadest reasonable constructions and their ordinary meanings as understood by those skilled in the art unless an explicit indication to the contrary in made herein. In particular, use of the singular articles such as “a,” “the,” “said,” etc. should be read to recite one or more of the indicated elements unless a claim recites an explicit limitation to the contrary. Further, regarding the methods and processes described herein, it should be understood that although the steps of such methods and processes have been described as occurring according to a certain ordered sequence, such processes could be practiced with the described steps performed in an order other than the order described herein. It further should be understood that certain steps could be performed simultaneously, that other steps (not described) could be added or implied, or that certain steps described herein could be omitted.

What is claimed is:

1. An implant for use in a female sterilization procedure comprising:
an implant body configured for insertion and placement within a fallopian tube, the implant body defining a proximal end and a distal end; and
a tether attached to the implant body and extending away from the implant body.

2. The implant of claim 1, wherein the tether extends at least about 5 mm from the implant body.

3. The implant of claim 2, wherein the tether extends between about 5 mm and about 10 mm from the implant body.

4. The implant of claim 1, wherein the tether is bioabsorbable.

5. The implant of claim 1, wherein the tether is attached to the proximal end of the implant body.

6. The implant of claim 1, where the tether is detachably coupled to the implant body and is adapted to detach from the implant body when a predetermined threshold force is applied to the tether.

7. The implant of claim 6, wherein the implant further includes a frangible interface between the implant body and the tether, and the tether is adapted to detach from the implant body at the frangible interface when a predetermined threshold force is applied to the tether.

8. The implant of claim 7, wherein the predetermined threshold force is between about 0.5 lbf and about 1.5 lbf.

9. The implant of claim 1, wherein the tether defines a first segment, a second segment longer than the first segment, a terminal end, and a frangible interface between the first and second segments, the first segment extending from the implant body to the frangible interface and the second segment extending from the frangible interface to the terminal end, wherein the second segment is adapted to detach from the first segment at the frangible interface when a predetermined threshold force is applied to the second segment.

10. The implant of claim 9, wherein the predetermined threshold force is between about 0.5 lbf and about 1.5 lbf.

11. The implant of claim 1, wherein the tether is a monofilament or multifilament suture.

12. The implant of claim 1, wherein the implant body comprises silicone.

13. The implant of claim 12, wherein the implant body is a substantially porous plug.

14. The implant of claim 13, wherein the implant body is substantially cylindrical shaped.

15. The implant of claim 14, wherein the implant body defines a longitudinal axis and the length of the implant body along the longitudinal axis is between about 2 mm and about 10 mm.
